(19) United States
(12) Patent Application Publication
(10) Pub. No.: US 2008/0293969 A1
(43) Pub. Date: Nov. 27, 2008

(54) 18F-Labeled Daa Analogues and Method of Labeling These Analogues as Positron Emission Tomography (Pet) Tracers For Imaging Peripheral Benzodiazepine Receptors

(75) Inventors: Farhad Karimi, Canton, MA (US); Bengt Langstrom, Uppsala (SE); Obaidur Rahman, Uppsala (SE); Sergio Estrada, Uppsala (SE)

Correspondence Address: GE HEALTHCARE, INC., IP DEPARTMENT, 101 CARNEGIE CENTER, PRINCETON, NJ, 08540-6231, US

(73) Assignee: GE Healthcare Limited, Amersham (GB)

(21) Appl. No.: 12/094,405

(22) PCT Filed: Nov. 20, 2006
(86) PCT No.: PCT/IB06/03281

§ 371 Date: May 21, 2008

Related U.S. Application Data
(60) Provisional application No. 60/739,085, filed on Nov. 22, 2005.

(51) Int. Cl. C07C 233/88

(52) U.S. Cl. 564/212

(57) ABSTRACT

Methods for selecting novel DAA analogues for a peripheral type benzodiazepine receptor were labeled with ¹⁸F using one-step syntheses are provided. Analogues labeled with the ¹⁸F using the one-step synthesis method are also provided. Additionally, the purification of the Br, I, Cl, TsO, MsO, or R_{f}SO₃ precursors in the compound of formula (II) by solid phase extraction is provided as is the precursor compounds of formula (II). A kit claim for comprising an effective amount of an ¹⁸F labeled compound, and pharmaceutically acceptable salts and solvates thereof are also provided.

### FIELD OF THE INVENTION

The present invention relates to new ¹⁸F-labeled DAA analogues and a method of labeling these analogues using a labeled precursor strategy. The resultant ¹⁸F-labeled DAA analogues are useful as Positron Emission Tomography (PET) tracers for imaging peripheral benzodiazepine receptors.

### BACKGROUND OF THE INVENTION

Tracers labeled with short-lived positron emitting radionuclides (e.g. ¹⁹F, t_{1/2}=110 minutes) are the positron-emitting nuclide of choice for many receptor imaging studies. Accordingly, radiolabeled ligands such as DAA1106 and its analogues have great clinical potential because of their utility in Positron Emission Tomography (PET) to quantitatively detect and characterize a wide variety of diseases.

Peripheral benzodiazepine receptors (PBR) are expressed in most organs and their expression is reported to be increased in activated microglia in the brain. PBR ligands such as [¹¹C]PK11195 have been widely used for the in vivo imaging of PBRs. The prototype PBR ligands such as Ro5-4864 and the isoquinoline carboxamide, PK11195, have been widely used to determine the cellular expression and function of PBR in various tissues wherein a ligand is defined herein said invention as a group, ion, or molecule coordinated to a central atom or molecule in a complex.

The [¹¹C]PK11195 ligand has long been used for imaging brain PBRs with Positron Emission Tomography (PET). However, this method has low sensitivity (i.e. a low ratio of PBR-specific to non-specific binding) and difficulty in quantization. Although [¹¹C]PK11195 is at present the ligand of choice, it is subject to shortcomings with respect to the contrast given between binding site and non-binding site expressing areas. In other words, the specific binding site of [¹¹C]PK11195 measured in the brain is only a small fraction of the total radioactivity. Therefore, recently a new class of high affinity PBR tracers that can measure the contrast between the binding and non-binding site has been developed based on aryloxyanilides.

Aryloxyanilides have shown promising results as ¹¹C radioligands for imaging PBRs. In the current invention, ¹⁸F-labeled analogues were developed based on aryloxynilides. ¹⁸F-labeled analogues are advantageous because they are produced in high activity typically 5-10 GBq, and due to the longer half-life of F-18, the labeled compound can be distributed to other sites for application.

Other compounds, however, such as N-(4-chloro-2-phenoxyphenyl)-N-(2-isopropoxybenzyl)-acetamide (DAA1097) and N-(2,5-dimethoxybenzyl)-N-(5-fluoro-2-phenoxyphenyl)acetamide (DAA1106) were reported as high affinity selective ligands for PBR at additional binding sites that Ro5-4864 and PK11195 were not. M. Cultry, P. Silver et al., Drug Dev. Research, 2001, vol. 52, 475-484. Moreover, PBR ligands such as Ro5-4864, PK11195, and DAA1106 have been labeled with ¹¹C. and used in PET studies, and in all cases a methylation method using [¹¹C] methyl iodide was used for labeling synthesis. M.-R. Zhang et al., Nucl. Med. Biol., 2003, vol. 30, 513-519. By using the [¹¹C] methyl iodide labeling of DAA1106 it is only possible to label the DAA1106 compound and not the analogues which are associated with an O-or N-methyl group.

Accordingly, there is a need for creating synthesis methods for ¹⁸F-analogues based on DAA-1106 as the lead compound. There is also a need to expand such methods to generate new compounds from the labeling of DAA1106 in order to find PET compounds for the study of the function of PBR in neuroinflammation and in inflammatory profiles within a patient wherein PBR is a key element of the steroidogenic pathway in peripheral tissues.

The term “analogue” used throughout this invention is defined as a chemical compound that is structurally similar to DAA1106 but differs in composition i.e. elements, functional groups. The term “tracer” used throughout this invention is defined as a substance used to trace the course of the chemical process.

Discussion or citation of a reference herein shall not be construed as an admission that such reference is prior art to the present invention.

### SUMMARY OF THE INVENTION

In view of the needs of the prior art, the present invention provides a method that relates to a one-step process for labeling ¹⁸F DAA1106 analogues for the study of the function of peripheral benzodiazepine receptors (PBR) in the neuroinflammation and anti-inflammatory profile of a patient.

The present invention depicts a method of preparing a compound of formula (I)

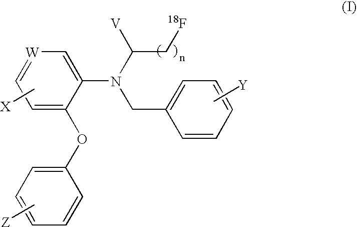

when the method comprises a one-step synthesis of a compound of formula (II)

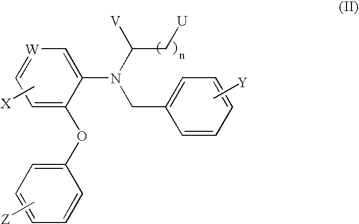

wherein U is any proper leaving group such as TsO, MsO, R_{f}SO₃, Br, I or Cl, and X and Z are independently H, F, Cl, Me, CF₃ or MeO and Y is H, MeO, Cl, EtO, n-PrO, i-PrO, n-PenO, i-PenO, MeS, n-Pr, CH₂═CH, OHC, MeCO, MeO₂C, (MeO)₂, and W is C or N, and n is 1, 2, or 3, and V is O or H₂
whereby U in the compound of formula (II) is the precursor for a high radiochemical yield in the range of about 85% to about 95%.

In a further embodiment, the compound of formula (I) is ¹⁸F-labeled N (2,5-dimethoxybenzyl)-3-fluoro-N-(5-fluoro-2-phenoxyphenyl) propanamide.

Yet another embodiment comprises of a method for preparing the compound of formula (I), wherein the compound of formula (II) is further purified by a solid phase extraction (SPE) method.

In a further embodiment of the present invention is a compound of formula (I),

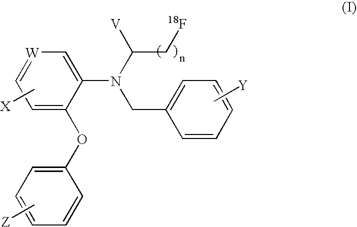

wherein U is TsO, MsO, R_{f}SO₃, Br, I or Cl, and X and Z are independently H, F, Cl, Me, CF₃, or MeO and Y is H, MeO, Cl, EtO, n-PrO, i-PrO, n-PenO, i-PenO, MeS, n-Pr, CH₂═CH, OHC, MeCO, MeO₂C, (MeO)₂, and W is C or N, and n is 1, 2, or 3, and V is O or H₂.

Yet in another embodiment of the invention, the compound of formula (I) is ¹⁸F-labeled N-(2,5-dimethoxybenzyl)-3-fluoro-N-(5-fluoro-2-phenoxyphenyl) propanamide.

In another embodiment of the invention, a compound of formula (II),

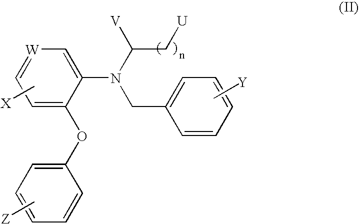

wherein U is Br, I, Cl, TsO, MsO, or R_{f}SO₃X and Z are independently H, F, Cl, Me,
CF₃, or MeO and Y is H, MeO, Cl, EtO, n-PrO, i-PrO, n-PenO, i-PenO, MeS, n-Pr,
CH₂═CH, OHC, MeCO, MeO₂C, (MeO)₂, and W is C or N, and n is 1, 2, or 3, and
   V is O or H₂ is claimed.

The present invention also provides for preparing a compound of formula (I) comprising an effective amount of a fluorine-isotope labeled compound, and pharmaceutically acceptable salts and solvates thereof, wherein the compound is ¹⁸F-labeled N-(2,5-dimethoxybenzyl)-3-fluoro-N-(5-fluoro-2-phenoxyphenyl) propanamide.

### DETAILED DESCRIPTION OF THE INVENTION

In the current invention, as mentioned previously, ¹⁸F-labeled analogues were developed based on aryloxynilides. Aryloxyanilides have showed promising results as radioligands for imaging peripheral type benzodiazepine binding site (PBR). Efficient ¹⁸F-labeled analogues have special value, since they can be produced in high activity and distributed to other nearby sites for application.

For example, the compound 3-bromo-N-(2,5-dimethoxybenzyl)-N-(5-fluoro-2-phenoxyphenyl) propanamide was synthesized in one-step using dry DMF to produce the novel compound ¹⁸F-labeled N-(2,5-dimethoxybenzyl)-3-fluoro-N-(5-fluoro-2-phenoxyphenyl) propanamide (SAN) with a high radiochemical yield of about 85% to about 95% using the corresponding bromide as a precursor. Furthermore, ¹⁸F-labeled SAN exhibited good binding and specificity for PBR-rich tissues as assessed in frozen section autoradiography. Additionally, the bromide precursor can be substituted with Cl, I, TsO, MsO, or the R_{f}SO₃ precursor. Furthermore, the DAA analogues along with its respective bromide, Cl, I, TsO, MsO, or the R_{f}SO₃ precursor can be further purified by solid phase extraction (SPE).

Additionally, after obtaining the ¹⁸F DAA analogues by the one-step method of synthesis, using an automated system termed FastLab or Tracerlab, high performance liquid chromatography (HPLC) is used to verify the structure of the analogues. A further tool was used to verify the structure of the analogues wherein a calculation study was conducted to look into the physical properties and 3D images of various analogues. The calculation study was conducted using a computer-aided molecular design modeling tool also know as CAChe. CAChe enables one to draw and model molecules as well as perform calculations on a molecule to discover molecular properties and energy values. The calculations are performed by computational applications, which apply equations from classical mechanics and quantum mechanics to a molecule. For example, a claimed novel compound such as ¹⁸F-labeled N-(2,5-dimethoxybenzyl)-3-fluoro-N-(5-fluoro-2-phenoxyphenyl) propanamide (SAN) was designed using CAChe.

There are several advantages for using this one-step synthetic method over previous processes. Inherent advantages are a slower reaction time and thus a faster synthesis of ¹⁸F-labeled compounds for production. Other advantages for using said claimed labeling method are the production of a higher yield of the novel ¹⁸F analogues due to avoiding side reactions, higher reproducibility of the ¹⁸F analogues, a convenient and easy to use synthetic procedure both manually and automated by systems such as FastLab, and the precursors such as Br, Cl, I, TsO, MsO, or R_{f}SO₃ might be easily prepared for marketing. Furthermore, using the R_{f}SO₃ precursor also offers potential simplifications of the overall process in going from ¹⁸F-fluoride in water to pure radio-pharmaceutical. In this case, a fluorous-SPE can be charged with the crude reaction mixture containing the ¹⁸F— labeled analogue and R_{f}SO₃ precursor. The pure ¹⁸F— labeled analogue can be eluted using fluorophobic solvent such as 80:20 MeOH— water.

Below a detailed description is given of a method for labeling ¹⁸F DAA analogues for a peripheral type benzodiazepine receptor by using a one-step synthesis for the study of the function of PBR in neuroinflammation and other inflammatory diseases in a patient.

In one embodiment of the present invention comprises preparing a compound of formula (I)

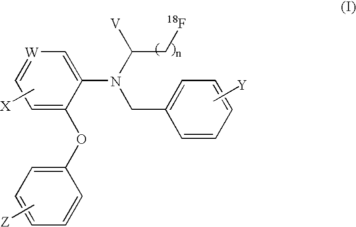

when the method comprises a one-step synthesis of a compound of formula (II)

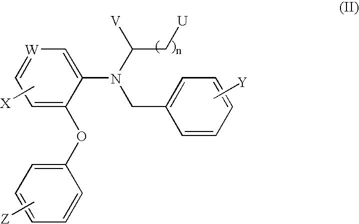

wherein U is Br, I, Cl, TsO, MsO, or R_{f}SO₃, and X and Z are independently H, F, Cl, Me, CF₃ or MeO and Y is H, MeO, Cl, EtO, n-PrO, i-PrO, n-PenO, i-PenO, MeS, n-Pr, CH₂═CH, OHC, MeCO, MeO₂C, (MeO)₂, and W is C or N, and n is 1, 2, or 3, and V is O or H₂
whereby U in the compound of formula (II) is the precursor for a high radiochemical yield in the range of about 85% to about 95% and the compound of formula (I) could overcome the contrast given between binding and nonbinding site expressing areas for imaging PBR in neurodegenerative and inflammatory diseases found in other tracers.

In a further embodiment, the compound of formula (I) is ¹⁸F-labeled N-(2,5-dimethoxybenzyl)-3-fluoro-N-(5-fluoro-2-phenoxyphenyl) propanamide.

Yet another embodiment comprises of a method for preparing the compound of formula (I), wherein the compound of formula (II) with a bromide, Cl, I, TsO, MsO, or R_{f}SO₃ is further purified by a solid phase extraction (SPE) method.

In a further embodiment of the present invention is a compound of formula

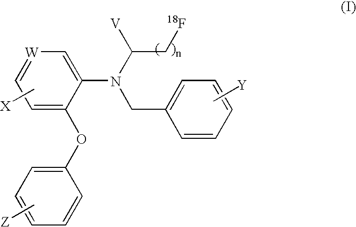

wherein U is Br, I, Cl, TsO, MsO, or R_{f}SO₃, and X and Z are independently H, F, Cl, Me, CF₃, or MeO and Y is H, MeO, Cl, EtO, n-PrO, i-PrO, n-PenO, i-PenO, MeS, n-Pr, CH₂═CH, OHC, MeCO, MeO₂C, (MeO)₂, and W is C or N, and n is 1, 2, or 3, and V is O or H₂.

Yet in another embodiment of the invention, the compound of formula (I) is ¹⁸F-labeled N-(2,5-dimethoxybenzyl)-3-fluoro-N-(5-fluoro-2-phenoxyphenyl) propanamide.

In another embodiment of the invention, a compound of formula (II),

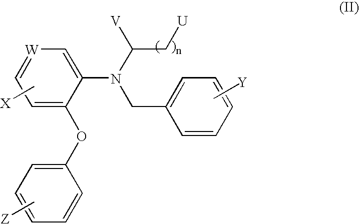

wherein U is Br, I, Cl, TsO, MsO, or R_{f}SO₃X and Z are independently H, F, Cl, Me, CF₃, or MeO and Y is H, MeO, Cl, EtO, n-PrO, i-PrO, n-PenO, i-PenO, MeS, n-Pr, CH₂═CH, OHC, MeCO, MeO₂C, (MeO)₂, and W is C or N, and n is 1, 2, or 3, and V is O or H₂ is claimed.

The present invention also provides for preparing a compound of formula (I) comprising an effective amount of a fluorine-isotope labeled compound, and pharmaceutically acceptable salts and solvates thereof, wherein the compound is ¹⁸F-labeled N-(2,5-dimethoxybenzyl)-3-fluoro-N-(5-fluoro-2-phenoxyphenyl) propanamide.

A further embodiment of the present invention comprises a kit for preparing a compound of formula (II), wherein U is Br, I, Cl, TsO, MsO, or R_{f}SO₃X and Z are independently H, F, Cl, Me, CF₃, or MeO and Y is H, MeO, Cl, EtO, n-PrO, i-PrO, n-PenO, i-PenO, MeS, n-Pr, CH₂═CH, OHC, MeCO, MeO₂C, (MeO)₂, and W is C or N, and n is 1, 2, or 3, and V is O or H₂.

Still another embodiment comprises a method of use for preparing a compound of formula (I)

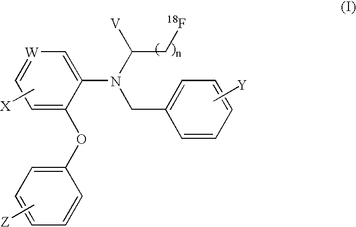

comprising an effective amount of a fluorine-isotope labeled compound, and pharmaceutically acceptable salts and solvates thereof, wherein the compound is ¹⁸F-labeled N-(2,5-dimethoxybenzyl)-3-fluoro-N-(5-fluoro-2-phenoxyphenyl) propanamide.

Yet another embodiment comprises a method of use for preparing a compound of formula (II),

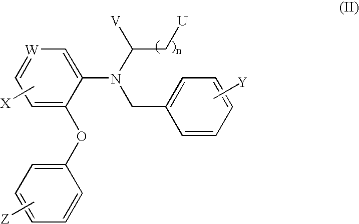

wherein U is Br, I, Cl, TsO, MsO, or R_{f}SO₃X and Z are independently H, F, Cl, Me, CF₃, or MeO and Y is H, MeO, Cl, EtO, n-PrO, i-PrO, n-PenO, i-PenO, MeS, n-Pr, CH₂═CH, OHC, MeCO, MeO₂C, (MeO)₂, and W is C or N, and n is 1, 2, or 3, and V is O or H₂.

A further embodiment of the present invention comprises a use of a compound of formula (I),

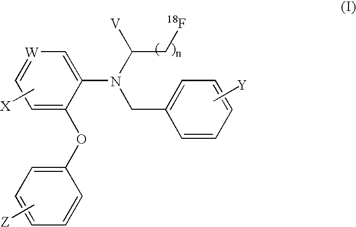

wherein X and Z are independently H, F, Cl, Me, CF₃, or MeO and Y is H, MeO, Cl, EtO, n-PrO, i-PrO, n-PenO, i-PenO, MeS, n-Pr, CH₂═CH, OHC, MeCO, MeO₂C, (MeO)₂, and W is C or N, andnis 1, 2, or 3, and V is O or H₂.

Still another embodiment of the present invention depicts the use of a compound of formula (II),

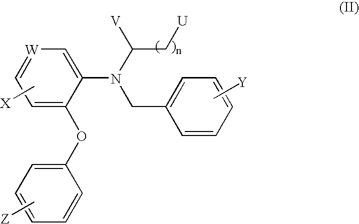

wherein U is Br, I, Cl, TsO, MsO, or R_{f}SO₃X and Z are independently H, F, Cl, Me, CF₃, or MeO and Y is H, MeO, Cl, EtO, n-PrO, i-PrO, n-PenO, i-PenO, MeS, n-Pr, CH₂═CH, OHC, MeCO, MeO₂C, (MeO)₂, and W is C or N, and n is 1, 2, or 3, and V is O or H₂.

### EXAMPLES

The invention is further described in the following examples which are in no way intended to limit the scope of the invention.

General Method for Preparing ¹⁸F

[¹⁸F] Fluoride was produced at Uppsala Imanet by the ¹⁸O(p, n) ¹⁸F nuclear reaction, wherein p stands for proton and n is neutron, through proton irradiation of enriched (95%) ¹⁸O water using Scanditronix MC-17 cyclotron which can produce 17 MeV protons.

### Example 1

### Experimental Studies

I. Precursor Synthesis

Preparation of 3-bromo-N-(2,5-dimethoxybenzyl)-N-(5-fluoro-2-phenoxyphenyl)propanamide

2,5-Dimethoxy benzyl-(5-fluoro-2-phenoxy)phenyl amine (0.30 g, 0.85 mmol) was dissolved in dry tetrahydrofuran (THF) (10 ml) and pyridine (0.12 ml, 1.47 mmol) was added. The mixture was cooled to 0° C. on an ice bath and 3-bromopropanoyl chloride (0.24 g, 0.14 ml, 1.40 mmol) was added slowly. The resulting mixture was allowed to warm at room temperature and stirred for 1 hour. The solvent was removed under reduced pressure and the residue was partitioned between water (100 ml) and EtOAc (200 ml). The organic layer was washed with brine (2×100 ml) and dried over MgSO₄. The solvent was removed under reduced pressure to obtain the crude product as light yellow oil. The crude product was used without further purification.

II. ¹⁸F-Labeling Synthesis

Preparation of the [K/K2.2.2]⁺¹⁸F⁻ (using enriched 95% ¹⁸O water)

After irradiation, the target content was passed through a pre-conditioned QMA cartridge resin. The column Was purged with helium for five minutes. The [¹⁸F]fluoride adsorbed on the resin was eluted into a reaction vial with 4 ml of a 96:4 (by volume) acetonitrile-water mixture containing 19.1 mg of kryptofix 2.2.2, wherein kryptofix 2.2.2 is a base transfer catalyst that transports the ¹⁸F-fluoride into the organic phase where the reaction take place, and 2.9 mg of K₂CO₃; the solution was then evaporated and co-evaporated with anhydrous acetonitrile (2×1 ml) to dryness in a nitrogen stream at 110° C. as shown below.

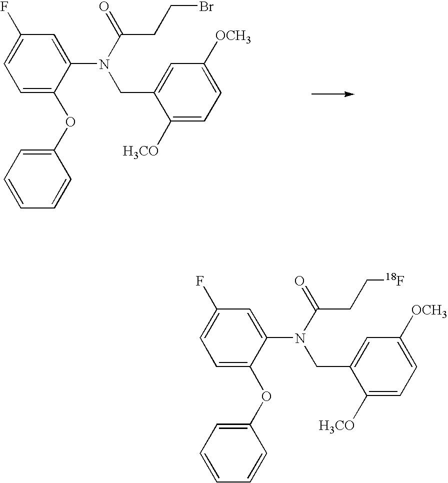

Preparation of the [¹⁸F] labeled N-(2,5-dimethoxybenzyl)-3-fluoro-N-(5-fluoro-2-phenoxy-phenyl)propanamide.

A solution of 3-bromo-N-(2,5-dimethoxybenzyl)-N-(5-fluoro-2 phenoxyphenyl) propanamide (4.4 mg) in anhydrous DMF (0.5 ml) was added to dry the [K/K2.2.2]⁺¹⁸F⁻. The reaction mixture was heated at 150° C. for 15 minutes. The crude mixture was analyzed and purified by analytical High Performance Liquid Chromotography (HPLC) in an isocratic elution of 20% KH₂PO₄ (25 mM) and 80% MeCN/H₂O (50:7), and a flow rate of 1.5 ml/min.

III. Preliminary Biological Validation

Frozen Section Autoradiography.

Cryosections of pig adrenal, rat heart and rat brain were incubated with [¹⁸F] labeled N-(2,5-dimethoxybenzyl)-3-fluoro-N-(5-fluoro-2-phenoxy-phenyl)propanamide (SAN1) for 40 min at R.T. in 50 mM Tris-HCl, pH 7.4, in the presence or absence of 1 μM PK11195 (selective PBR ligand). Thereafter, sections were washed in buffer 3×1 min, dried in a heated oven (37° C.) and exposed to phosphor imaging plates for 240 min. Assessment of the selectivity of the radioligand for PBRs was assessed by comparing the regional distribution of radioactivity with known distribution of PBRs in tissue. Another parameter investigated was the blocking effect in sections which were co-incubated with an excess of PK11195. SAN1 showed promising properties with high uptake in adrenal medulla, which was completely blocked by an excess of PK11195, as illustrated below (A shows sections incubated with SAN1, while sections in B have been co-incubated with 1 μM PK11195.

Biodistribution in Rats.

SAN1 at 10 MBq/kg was injected in the tail vein of male Sprague-Dawley rats. Rats were sacrificed 15 min post-injection and blood, cortex, olfactory bulb, adrenal and cerebellum were dissected, weighed and radioactivity in the organs was measured. Uptake in organs was expressed as Standardized Uptake Value, SUV. The scope of this experiment was to assess blood brain penetration of the radiolabeled compound as well as its distribution to organs known to express PBR, e.g. adrenal medulla. Results were encouraging as SAN1 showed a brain to blood ratio well above 1 in all regions analyzed, particularly in olfactory bulb, which has been reported to be one structure with higher expression of PBR in non-diseased brain. Additionally, we observed a large uptake of SAN1 in adrenal which is the peripheral organ with the highest density of PBRs. Biodistribution data from 4 rats was summarized and is shown in the diagram below, error bars represent the SEM.

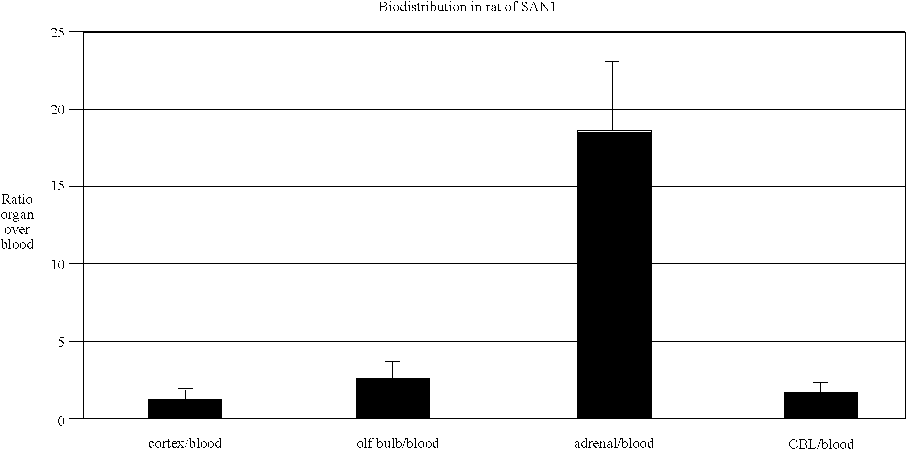

### SPECIFIC EMBODIMENTS, CITATION OF REFERENCES

The present invention is not to be limited in scope by specific embodiments described herein. Indeed, various modifications of the inventions in addition to those described herein will become apparent to those skilled in the art from the foregoing description and accompanying figures. Such modifications are intended to fall within the scope of the appended claims.

Various publications and patent applications are cited herein, the disclosures of which are incorporated by reference in their entireties.

What is claimed is:

1. A method for preparing a compound of formula (I) 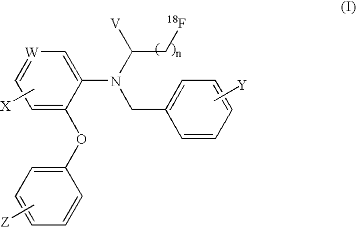
when the method comprises a one-step synthesis of a compound of formula (II) 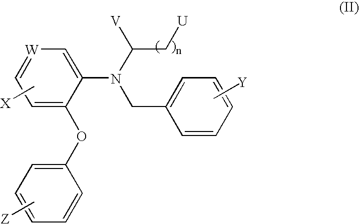
wherein U is Br, I, Cl, TsO, MsO, or R_{f}SO₃, and X and Z are independently H, F, Cl, Me, CF₃ or MeO and Y is H, MeO, Cl, EtO, n-PrO, i-PrO, n-PenO, i-PenO, MeS, n-Pr, CH₂═CH, OHC, MeCO, MeO₂C, (MeO)₂, and W is C or N, and n is 1, 2, or 3, and V is O or H₂
whereby the compound of formula (I) could overcome the contrast given between binding and nonbinding site expressing areas for imaging peripheral benzodiazepine receptors (PBR) in neurodegenerative and inflammatory diseases found in other tracers.

2. The method for preparing the compound of formula (I) according to claim 1, wherein the compound of formula (I) is ¹⁸F-labeled N-(2,5-dimethoxybenzyl)-3-fluoro-N-(5-fluoro-2-phenoxyphenyl) propanamide.

3. The method for preparing the compound of formula (I) according to claim 1, wherein the compound of formula (II) is further purified by a solid phase extraction (SPE) method.

4. A compound of formula (I), 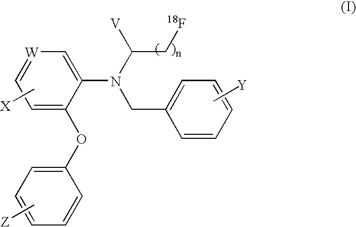
wherein X and Z are independently H, F, Cl, Me, CF₃, or MeO and Y is H, MeO, Cl, EtO, n-PrO, i-PrO, n-PenO, i-PenO, MeS, n-Pr, CH₂═CH, OHC, MeCO, MeO₂C, (MeO)₂, and W is C or N, and n is 1, 2, or 3, and V is O or H₂.

5. The compound of formula (I) according to claim 4, wherein the compound of formula (I) is ¹⁸F-labeled N-(2,5-dimethoxybenzyl)-3-fluoro-N-(5-fluoro-2-phenoxyphenyl) propanamide.

6. A compound of formula (II), 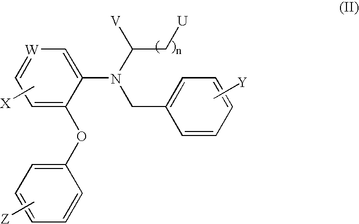
wherein U is Br, I, Cl, TsO, MsO, or R_{f}SO₃X and Z are independently H, F, Cl, Me, CF₃, or MeO and Y is H, MeO, Cl, EtO, n-PrO, i-PrO, n-PenO, i-PenO, MeS, n-Pr, CH₂═CH, OHC, MeCO, MeO₂C, (MeO)₂, and W is C or N, and n is 1, 2, or 3, and V is O or H₂.

7. A kit for preparing a compound of formula (I) comprising an effective amount of a fluorine-isotope labeled compound, and pharmaceutically acceptable salts and solvates thereof, wherein the compound is ¹⁸F-labeled N-(2,5-dimethoxybenzyl)-3-fluoro-N-(5-fluoro-2-phenoxyphenyl) propanamide.

8. A kit for preparing a compound of formula (II), wherein U is Br, I, Cl, TsO, MsO, or R_{f}SO₃X and Z are independently H, F,
Cl, Me, CF₃, or MeO and Y is H, MeO, Cl, EtO, n-PrO, i-PrO, n-PenO, i-PenO, MeS, n-Pr, CH₂═CH, OHC, MeCO, MeO₂C, (MeO)₂, and W is C or N, and n is 1, 2, or 3, and V is O or H₂.

9. A method of use for preparing a compound of formula (I) comprising an effective amount of a fluorine-isotope labeled compound, and pharmaceutically acceptable salts and solvates thereof, wherein the compound is ¹⁸F-labeled N-(2,5-dimethoxybenzyl)-3-fluoro-N-(5-fluoro-2-phenoxyphenyl) propanamide.

10. A method of use for preparing a compound of formula (II), wherein U is Br, I, Cl, TsO, MsO, or R_{f}SO₃X and Z are independently H, F, Cl, Me, CF₃, or MeO and Y is H, MeO, Cl, EtO, n-PrO, i-PrO, n-PenO, i-PenO, MeS, n-Pr, CH₂═CH, OHC, MeCO, MeO₂C, (MeO)₂, and W is C or N, and n is 1, 2, or 3, and V is O or H₂.

11. Use of a compound of formula (I), 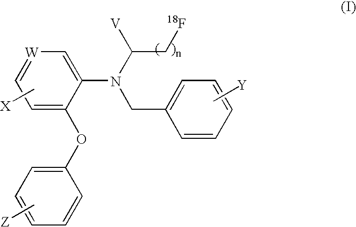
wherein X and Z are independently H, F, Cl, Me, CF₃, or MeO and Y is H, MeO, Cl, EtO, n-PrO, i-PrO, n-PenO, i-PenO, MeS, n-Pr, CH₂═CH, OHC, MeCO, MeO₂C, (MeO)₂, and W is C or N, and n is 1, 2, or 3, and V is O or H₂.

12. Use of a compound of formula (II), 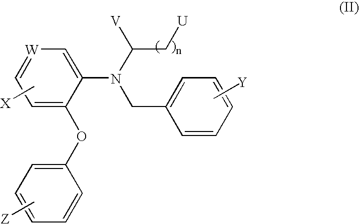
wherein U is Br, I, Cl, TsO, MsO, or R_{f}SO₃X and Z are independently H, F, Cl, Me, CF₃, or MeO and Y is H, MeO, Cl, EtO, n-PrO, i-PrO, n-PenO, i-PenO, MeS, n-Pr, CH₂═CH, OHC, MeCO, MeO₂C, (MeO)₂, and W is C or N, and n is 1, 2, or 3, and V is O or H₂.
